(19) United States
(12) Patent Application Publication
(10) Pub. No.: US 2023/0111779 A1
(43) Pub. Date: Apr. 13, 2023

(54) METHODS OF MANUFACTURE OF DIANHYDRIDES

(71) Applicant: Mani Raj Chaulagain, San Diego, CA (US)

(72) Inventor: Mani Raj Chaulagain, San Diego, CA (US)

(21) Appl. No.: 18/062,189

(22) Filed: Dec. 6, 2022

Related U.S. Application Data
(63) Continuation-in-part of application No. PCT/US2021/033960, filed on May 25, 2021.
(60) Provisional application No. 63/036,486, filed on Jun. 9, 2020.

(51) Int. Cl. C07D 493/00

(52) U.S. Cl. CPC C07D 493/00

(57) ABSTRACT

A method of making diimide and dianhydride that includes contacting a nitro or halo N-substituted phthalimide with bisphenol in polar aprotic solvents, such as dimethylsulfoxide and sodium hydride to provide high conversion to a diimide; precipitating the product in acetic acid solution and filtration; treating the resulting solid, N-substituted diimide with a carboxylic acid and substituted or unsubstituted dimethyl sulfoxide in an aqueous medium to provide a reaction mixture including tetra acid, triacid, imide diacid and diimide along with substituted or unsubstituted acetic acid, dimethyl sulfoxide and their derivatives. The method includes the isolation of tetra acid by precipitation in water followed by centrifuge or filtration. The tetra acid is converted into the corresponding dianhydride. The dianhydride prepared by the method are also described as precursor to make polyetherimide.

### PRIORITY STATEMENT

The present application:
is a Continuation-in-Part Application of PCT Application PCT/US21/033960, filed May 25, 2021;
which claims benefit of priority to U.S. Provisional Application Serial No: 63/036,486, filed Jun. 9, 2020;
each of which is incorporated by reference herein in its entirety.

### TECHNICAL FIELD

The present invention relates generally to the fields of processes for making diimides and dianhydrides which are intermediates for the manufacture of polyetherimides.

### BACKGROUND

Diimides and dianhydrides are the key intermediates for manufacturing polyimides (PIs), especially polyetherimides (PEIs). PEIs are amorphous and transparent high-performance polymers with glass transition temperatures greater than 180° C. These polymers are known for possessing high strength, heat resistance, modulus, and broad chemical resistance. Due to these features, PEIs are widely used in diverse applications such as automotive, telecommunication, aerospace, electronics/electrical, transportation and healthcare. Polyetherimides are manufactured by condensation polymerization of dianhydrides and diamines. The dianhydrides are manufactured in various ways. Making dianhydrides from diimides is one of the most commonly used processes. For example, dianhydrides can be made from aromatic diimides such as N-substituted bisphenol A diimides (5,5′-((propane-2,2-diylbis(4,1-phenylene))bis(oxy))bis(2-methylisoindoline-1,3-dione)), which has the following structure.

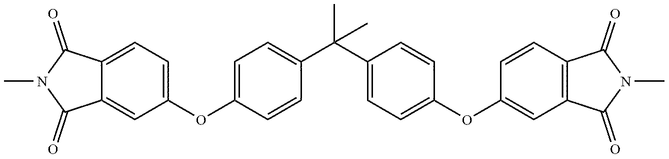

Other variants of the diimides can also be present. Diimides such as 1, in turn, can be produced by displacement reactions typically carried out between a bisphenols such as bisphenol-A or biphenol with substituted phthalimides such as nitro or halo N- substituted phthalimide with the help of a base. The conventional process of making diimides involves making the bisphenol salt as an aqueous solution followed by drying the salt and displacement reaction with nitro or halo N-substituted phthalimide with the help of catalyst.

Conventionally, the conversion of diimides to dianhydrides is typically carried out by two main processes. One process involves a two-step protocol; the alkaline hydrolysis of diimide followed by acidification to make tetra acid which is then ring closed to make dianhydride. Another process involves the exchange reaction of diimide with phthalic anhydride in aqueous medium in the presence of triethylamine to form tetra acid salt which is then ring closed to produce the dianhydride. The later process is the incomplete conversion of diimide to dianhydride which requires the extraction with organic solvent to purify the tetra acid salt and recycling of the unreacted diimide and other byproducts.

Simon Padmanabhan in WO 2019/245898 A1, Aaron Royer in WO 2019/222077 A1 and WO 2017/189293 A1, Robert Werling in WO 2019/236536 A1, Gregory Hemmer in WO 2019/217257 A1, Jimmy Webbs in US 4,329,496 and US 4,318,857, Brent Dellacoletta in US 6,008,374 and US 5,536,846, Darrel Heath in US 3,879,428 and US 3,957,862 and James Silva in US 4,571,425, generally report the synthesis of dianhydride from diimide by the exchange reaction in aqueous media. However, their methods are low yielding and require isolation and recycling of materials using organic solvents at high temperature and pressure. James Schulte in WO 2017/172593 A1 generally reports the synthesis of dianhydride from diimide. However, their methods use multiple step protocols of alkaline hydrolysis and acidification and do not relate to the reagents and protocol used in the present invention. On the other hand, diimide synthesis traditionally involves making the bisphenol salt in aqueous medium, drying and displacement using catalyst.

Thus, there remains a need for an improved and convenient method for the manufacturing and isolating diimides without the need of drying and catalyst. Also, there is a need of manufacturing and isolating dianhydrides from diimides in a single step that can provide high yields and do not require extraction process for purification and also avoids multi-step alkaline hydrolysis followed by acidification protocol.

### SUMMARY

The present invention recognizes that there exists a long felt need for methods of the synthesis of diimide and dianhydride, and the products of those processes as well.

A first aspect of the present invention generally relates to a method for the synthesis of a diimide and dianhydride.

A second aspect of the present invention generally relates to diimide and dianhydride made by a method of the present invention.

### DETAILED DESCRIPTION OF THE PRESENT INVENTION

### Definitions

Unless defined otherwise, all technical and scientific terms used herein have the same meaning as commonly understood by one of ordinary skill in the art to which the present invention belongs. Generally, the nomenclature used herein and the laboratory procedures in cell culture, chemistry, microbiology, molecular biology, cell science and cell culture described below are well known and commonly employed in the art. Conventional methods are used for these procedures, such as those provided in the art and various general references such as but not limited to WO 2019/245898 A1 and WO 2017/172593 A1. Where a term is provided in the singular, the inventors also contemplate the plural of that term; and where a term is provided in the plural, the inventors also contemplate the singular of that term. The nomenclature used herein and the laboratory procedures described below are those well-known and commonly employed in the art. As employed throughout the disclosure, the following terms, unless otherwise indicated, shall be understood to have the following meanings:

All ranges disclosed herein are inclusive of the endpoints, and the endpoints are independently combinable with each other. “Combinations” is inclusive of blends, mixtures, alloys, reaction products, and the likes. The terms “first”, “second”, and like, do not denote any order, quantity, or importance, but rather are used to donate one element from other. The terms “a”, “an”, and “the” do not denote a limitation of quantity, and are to be constructed to cover both the singular and the plural, unless otherwise indicated herein or clearly contradicted by context. “Or” means “and/or” unless clearly stated otherwise. Reference throughout the specification to “some embodiments”, “an embodiment”, “some aspects”, “an aspect”, and so forth, means that a particular element described in connection with the embodiment or aspect is included in at least one embodiment or aspect described herein, and may or may not be present in other embodiments or aspects. In addition, it is to be understood that the described elements may be combined in any suitable manner in the various embodiments or aspects.

Unless defined otherwise, technical and scientific terms used herein have the same meaning as is commonly understood by one of the skills in the art to which this application belongs. All cited patents, patents applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position substituted by any indicated group is understood to have its valency filled by a bond as indicated or a hydrogen atom. A dash (“-”) that is not between two letters or symbols is used to indicate a point of attachment for a substituent. For example, —CHO, is attached to the carbon of the carbonyl group.

The term “hydrocarbyl”, whether used by itself or as a prefix, suffix, or fragment of another term, refers to a residue that contain only carbon and hydrogen. The residue can be aliphatic or aromatic, straight-chain, cyclic, bicyclic, branched, saturated, or unsaturated. It can also contain combinations of aliphatic, aromatic, straight-chain, cyclic, bicyclic, branched, saturated, and unsaturated hydrocarbon moieties. However, when the hydrocarbyl residue is described as substituted, it may, optionally, contain heteroatoms over and above the carbon and hydrogen members of the substituent residue. Thus, when specifically described as substituted, the hydrocarbyl residue can also contain one or more carbonyl groups, amino groups, hydroxyl groups, or the like, or it can contain heteroatoms within the backbone of the hydrocarbyl residue. The term “alkyl” means a branched or straight-chain, saturated aliphatic hydrocarbon group, e.g., methyl, ethyl, n-propyl, iso-propyl, n-butyl, sec-butyl, tert-butyl, n-pentyl, sec-pentyl, n-hexyl, and sec-hexyl. The term “alkenyl” means a straight-chain or branched-chain, monovalent hydrocarbon group having at least one carbon-carbon double bond. The term “alkoxy” means an alkyl group that is linked via an oxygen, for example methoxy, ethoxy, and sec-butoxy groups.

While particular embodiments and aspects have been described, alternative, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents. The term “alkylene” means a straight or branched chain, saturated, divalent aliphatic hydrocarbon group (e.g., methylene (—CH₂—), or ethylene (—CH₂CH₂—)). Cycloalkylene means a divalent cyclic alkylene group, -CₙH₂ₙ₋₂-. “Cycloalkenyl” means a monovalent group having one or more rings and one or more carbon-carbon double bonds in the ring, wherein all ring members are carbon (e.g., cyclopentenyl, cyclohexenyl). “Aryl” means an aromatic hydrocarbon group containing the specified number of carbon atoms, such as phenyl, troponyl, indanyl, or naphthyl. “Arylene” means divalent aryl group. “Arylalkylene” means an arylene group substituted with an alkyl group. The prefix “halo” means one or more of a fluoro-, chloro-, bromo-, or iodo- substituent in a group or compound. The prefix “hetero” means that the compound or a group containing heteroatoms N, O, S, P, or Si. “substituted” means that the compound or group is substituted with at least one (e.g., 1, 2, 3, or 4) substituents that can each independently be a C₁₋₉ alkoxy, a C₁₋₉ haloxy, a nitro (—NO₂), cyano (—CN), a C₁₋₆ alkyl sulfonyl (-SO₂-alkyl), a C₆₋₁₂ aryl sulfonyl (-SO₂-aryl), a thiol (—SH), a thiocyano (—SCN), a tosyl (CH₃C₆H₄SO₂-), a C₃₋₁₂ cycloalkyl, a C₅₋₁₂ cycloalkenyl, a C₆₋₁₂ aryl, a C₇₋₁₃ arylalkylene, a C₄₋₁₂ heterocycloalkyl, and a C₃₋₁₂ heteroaryl instead of hydrogen, provided that the substituted atom’s normal valence is not exceeded. The number of carbon atoms indicated in a group is exclusive of any substituents. For example —CH2CH₂CN is a C₂ alkyl group substituted with a nitrile group.

“Directly” refers to direct causation of a process that does not require intermediate steps.

“Indirectly” refers to indirect causation that requires intermediate steps.

Other technical terms used herein have their ordinary meaning in the art that they are used, as exemplified by a variety of technical dictionaries.

### INTRODUCTION

The present invention recognizes that there exists a long-felt need for methods of the synthesis of a diimide and dianhydride, and the products of those processes as well.

As a non-limiting introduction to the breath of the present invention, the present invention includes several general and useful aspects, including:
1) A method for the synthesis of a diimide and dianhydride; and
2) a diimide and dianhydride made by a method of the present invention.

These aspects of the present invention, as well as others described herein, can be achieved by using the methods, articles of manufacture and compositions of matter described herein. To gain a full appreciation of the scope of the present invention, it will be further recognized that various aspects of the present invention can be combined to make desirable embodiments and aspects of the present invention.

### I Methods of Making Diimide and Dianhydride Compositions

The present invention includes a method for the synthesis of a diimide and dianhydride composition.

Generally, the method for the synthesis of a diimide and dianhydride composition of the present invention includes contacting a nitro or halo N-substituted phthalimide with bisphenol in polar aprotic solvents, such as dimethylsulfoxide, with sodium hydride under conditions effective to provide high conversion to a diimide, wherein the reacting is at a reaction temperature that is between about 0 to about 250° C.; precipitating the product in acetic acid solution and filtration; treating the resulting solid, N-substituted diimide with an organic carboxylic acid in an aqueous medium with substituted or unsubstituted dimethyl sulfoxide under conditions effective to provide an aqueous reaction mixture including high conversion to a tetra acid along with triacid and an imide diacid, wherein the reacting is at a reaction temperature that is about 150 to about 250° C. and a reaction pressure of about 150 to about 300 psig; precipitating the products in water; and converting the tetra acid into the corresponding dianhydride by heating or any other conventional method.

The present invention provides methods for conversion of nitro- or halo N-substituted phthalimide to diimides and then dianhydrides. In particular, present inventor have found that the use of sodium hydride in polar aprotic solvents such as dimethyl sulfoxide can convert nitro or halo N-substituted phthalimide and bisphenol into diimide in high yields; which with the use of substituted or unsubstituted acetic acid and substituted or unsubstituted dimethyl sulfoxide in aqueous medium can be converted into tetra acids directly in high yields which can be isolated by precipitating in water and the precipitate tetra acid can be ring closed into dianhydride by heating.

The method includes reacting nitro or halo N-substituted phthalimide with bisphenol in polar aprotic solvents with sodium hydride under conditions effective to provide high conversion to a diimide followed by reacting the resulting diimide with a substituted or unsubstituted acetic acid and a substituted or unsubstituted dimethyl sulfoxide in an aqueous medium under the conditions effective to provide an aqueous reaction mixture.

Conventionally, the conversion has been carried out by making salt of bisphenol in aq medium followed by drying and then treating with nitro or halo substituted N-methylphthalimide to afford diimide which is then treated with phthalic anhydride, water and triethylamine resulting up to about 80% conversion. That required multi-step protocol to make diimide and purification protocol for dianhydride involving solvent extraction using flammable organic solvents under high temperature and pressure.

Another conventional method involves multi-step protocol to make diimide and multi-step protocol of alkaline hydrolysis followed by acidification and possible purification in each step and ring closing to make the dianhydrides.

The starting material phthalimide can be of formula (2)

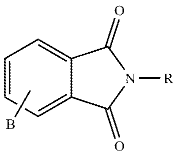

wherein, B is —NO2, —Cl, —F, —Br, or —I.

The starting material bisphenol can be of formula (3)

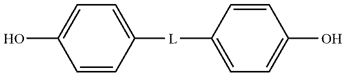

The intermediate diimide can be of the formula (4)

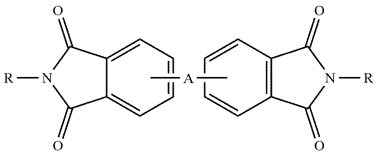

wherein A is —O—, —S—, —C(O)—, —SO₂—, —SO—, -C_{y}H_{2y}- wherein y is an integer from 1 to 5 or a halogenated derivative thereof or —O—E—O—, wherein E is an aromatic C₆₋₂₄ monocyclic or polycyclic moiety optionally substituted with 1 to 6 of C₁₋₈ alkyl groups, 1 to 8 halogen atoms, or a combination including at least one of the foregoing.

In an aspect of the present invention, the R is a monovalent C₁₋₁₃ organic group.

In an aspect of the present invention, the group A in the formula (4) is a substituted or unsubstituted divalent organic bond of the —O— or the —O—E—O— groups are in the 3,3’, 3,4’, 4,3’, and 4,’4 positions. Exemplary groups E include groups of formula (5):

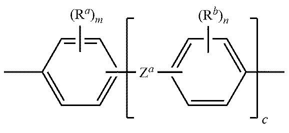

wherein R^{a} and R^{b} are each independently, a halogen atom or a monovalent C₁₋₆ alkyl group, and can be the same or different; m and n are each independently integers of 0 to 4; c is 0 to 4, specifically 0 or 1; and Z^{a} is a bridging group connecting the two aromatic groups, where the bridging group and point of attachment of each C₆ arylene group are disposed ortho, meta, or para (specifically para) to each other on the C₆ arylene group. The bridging group Z^{a} can be a single bond, —O—, —S—, —S(O)—, —S(O)2—, —C(O)—, or a C₁₋₁₈ organic bridging group. The C₁₋₁₈ organic bridging group can be cyclic or acyclic, aromatic or non-aromatic, and can further include heteroatoms such as halogens, oxygen, nitrogen, sulfur, silicon, or phosphorus. The C₁₋₁₈ organic group can be disposed such that the C₆ arylene groups connected thereto are each connected to a common alkylidene carbon or to different carbons of the C₁₋₁₈ organic bridging group. A specific example of a group E is a divalent group of formula (6)

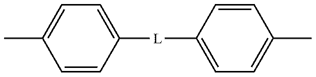

wherein L is a single bond, —O—, —S—, —C(O)—, —SO₂—, —SO—, —P(Ra)═O)— wherein R^{a} is a C₁₋₈ alkyl or C₆-₁₂ aryl or —C_{y}H_{2y}— wherein y is an integer from 1 to 5 or a halogenated derivative thereof (including a perfluoroalkylene group). Exemplary dihydroxy aromatic compounds from which E can be derived include but not limited to 2,2-bis-(2-hydroxyphenyl)propane, 2,4′-(dihydroxydiphenylmethane, bis(2-hydroxyphenyl)methane, 2,2-bis-(4-hydroxyphenyl)propane (also called bisphenol A or BPA), 1,1-bis-(4-hydroxyphenyl)ethane, 1,1-bis-(4-hydroxyphenyl)propane, 2,2-bis-(4-hydroxyphenyl)pentane, 3,3-bis-(4-hydroxyphenyl)pentane, 4,4′-dihydroxybiphenyl, 4,4′-dihydroxy-3,3,5,5′-tetramethylbiphenyl, 2,4′-hydroxybenzophenone, 4,4′-dihydroxydiphenylsulfone, 2,4′-dihydroxydiphenylsulfone, 4,4′-dihydroxydiphenylsulfoxide, 4,4′-dihydroxydiphenylsulfide, hydroquinone, resorcinol, 3,4-dihydroxydiphenylmethane, 4,4′-dihydroxybenzophenone, 4,4′-dihydroxydiphenylether, and the like, or a combination of including at least one of the forgoing.

In an aspect of the present invention, E is derived from bisphenol A, such that L in the above formula is 2,2-isopropylidene.

Thus in an aspect of the present invention, E is 2,2-(4-phenylene)isopropylidene (7).

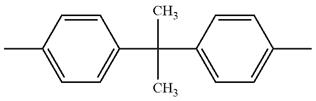

In an aspect of the present invention, E is derived from biphenol, such that L in the above formula is a single bond.

Thus in an aspect of the present invention, E is 4-phenylene-1,1′-biphenyl (8)

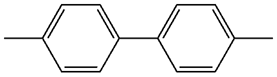

In an aspect of the present invention, R is a phenyl group, or C₁₋₄ alkyl group, for example a methyl group, an ethyl group, propyl group, or a butyl group, preferably a methyl group.

In an aspect of the present invention, the diimide including 4,4′-bisphenol A bis-N-methylphthalimide, 3,4′-bisphenolA-bis-N-methylphthalimide, 3,3′-bisphenolA-bis-N-methylphthalimide, 4,4′-biphenol bis-N-methylphthalide, 3,3′-biphenol-bis-N-methylphthalimide or a combination including at least one of the forgoing.

The carboxylic acid can be of the formula

X-COOH

wherein X is substituted or unsubstituted phenyl, a hydrogen, a monovalent C₁₋₆ alkyl group, a halogen substituted alkyl group, or a halogen.

In an aspect of the present invention, halo N-substituted phthalimide is Chloro N-methylphthalimide

In an aspect of the present invention, nitro N-substituted phthalimide is nitro N-methylphthalimide.

In an aspect of the present invention, bisphenol is Bisphenol A

In an aspect of the present invention, bisphenol is Biphenol

In an aspect of the present invention carboxylic acid is preferably acetic acid.

In an aspect of the present invention, the substituted or unsubstituted acetic acid is preferably acetic acid.

The substituted or unsubstituted dimethyl sulfoxide can be of formula

J₂SO

wherein two J can be same or different. J is a substituted or unsubstituted phenyl, a hydrogen, a monovalent C₁₋₅ alkyl group, or a halogen substituted alkyl group.

In an aspect of the present invention, the substituted or unsubstituted dimethyl sulfoxide is preferably dimethyl sulfoxide.

Reacting nitro or halo substituted N-substituted phthalimide with bisphenol in polar aprotic solvents with sodium hydride under conditions effective to provide high conversion to a diimide.

The reacting is further carried out under the conditions effective to provide a reaction mixture. Effective conditions can include reacting at a reaction temperature of between 0 to 250° C., such as but not limited to between about 0 to about 180° C. under inert atmosphere.

In an aspect of the present invention, the initial molar ratio of bisphenol; halo or nitro N-substituted phthalimide; and sodium hydride is between about 1:1.8:1.8 to about 1:2.2:2.2.

In an aspect of the present invention, the initial mass ratio of bisphenol and polar aprotic solvent is preferably between about 1:2 to about 1:20.

In another aspect of the present invention, the reacting the diimide with substituted or unsubstituted acetic acid and substituted or unsubstituted dimethyl sulfoxide is carried out in aqueous medium.

The reacting is further carried out under conditions effective to provide an aqueous reaction mixture. Effective conditions can include reacting at a reaction temperature of between about 150 to about 250° C., such as between about 160 to about 210° C., and a reaction pressure that is between about 160 to about 300 psig, such as between about 180 to about 240 psig.

In an aspect of the present invention, the initial mass ratio of acetic acid to diimide is between about 1:1 to about 10:1.

In an aspect of the present invention, the initial mass ratio of dimethyl sulfoxide to diimide is between about 1:1 to about 10:1.

In an aspect of the present invention, the initial aqueous reaction mixture is of less than about 10% wt., less than about 15% wt., less than about 20% wt., or less than about 25% wt.

The aqueous reaction resulted from the reaction of diimide with the substituted or unsubstituted acetic acid and substituted or unsubstituted dimethyl sulfoxide includes a tetra acid, at least one triacid, and imide diacid.

In an aspect of the present invention, tetra acid is of the formula

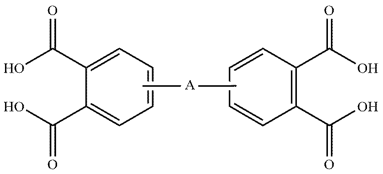

The triacid is of formula

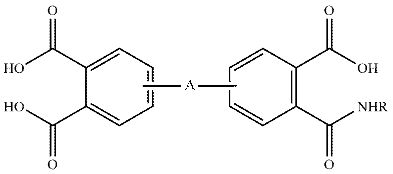

The imide diacid is of formula

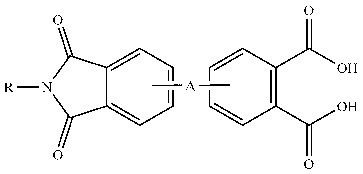

wherein A can be as described above, and preferably —O—, —S—, —C(O)—, —SO2—, —SO—, —CyH2y—wherein y is an integer from 1 to 5 or a halogenated derivative thereof or —O—E—O—, wherein E is an aromatic C₆₋₂₄ monocyclic or polycyclic moiety optionally substituted with 1 to 6 of C₁₋₈ alkyl groups, 1 to 8 halogen atoms, or a combination including at least one of the foregoing. R is a phenyl group, or C₁₋₄ alkyl group, for example a methyl group, an ethyl group, propyl group, or a butyl group, preferably a methyl group.

In an aspect of the present invention, A is —O—E—O—, wherein E is derived from bisphenol A or biphenol. The divalent bonds of the —O—E—O— groups are in the 3,3′, 3.4′, 4,3′, or the 4,4′ positions.

In an aspect of the present invention, the reaction mixture can further include nitro or halo N-substituted phthalimide, mono-imide, and diimide.

The method further includes isolating the diimide by precipitating the reaction mixture in water.

The method further includes the conversion of diimides into dianhydride in aqueous media.

In an aspect of the present invention, the aqueous reaction mixture can further include diimide.

Without wishing to be bound by theory, the reaction mixture can further contain acetic acid and its derivatives derived from the reaction, and the substituted and unsubstituted dimethyl sulfoxide, the derivatives of substituted or unsubstituted dimethyl sulfoxide, and decomposition products of substituted or unsubstituted dimethyl sulfoxides derived from the reaction.

The method of the present invention further includes isolating the tetra acid, containing the mixture of triacid, imide diacid and diimide by precipitating the reaction mixture at lower temperature in water.

In an aspect of the present invention, the volumetric ratio of added water and reaction mixture is between about 10:1 to about 0:1.

In an aspect of the present invention, the precipitation is carried out at temperature of between about 5° C. to about 50° C.

In an aspect of the present invention, the precipitation can be carried out without adding water at temperature of between about 5° C. to about 50° C.

The method further includes removing the aqueous phase by filtration or centrifuge of the aqueous slurry to obtain the powder cake of the mixture of the tetra acid, triacid, and imide diacid and diimide.

The method further includes making the solution of tetra acid in dimethyl sulfoxide as a varnish after removing water and acetic acid under reduced pressure

The method further includes converting the tetra acid into the corresponding dianhydride. Converting the tetra acid into the corresponding dianhydride can be readily determined by ordinary skill in the art such as a cyclization process with the formation of water.

In an aspect of the present invention, the precipitate of tetra acid is converted into dianhydride by heating at temperature of between about 140° C. to about 220° C. at pressure less than about 200 mm of Hg. Alternatively, the tetra acid can be converted into the dianhydride by refluxing in the presence of a dehydrating agent, such as acetic anhydride.

In an aspect of the present invention, the crude reaction mixture of tetra acid is converted into dianhydride by heating at temperature of between about 140° C. to about 220° C. at pressure less than about 200 mm of Hg.

The dianhydride can be used to make polyimides, especially polyetherimides.

Polyetherimides can be prepared by any of the well-known skill in the art. The common method of making polyetherimides from dianhydrides is the reaction of the dianhydride of formula (12)

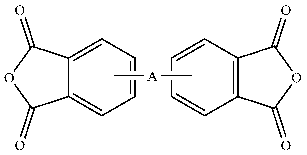

with a diamine of the formula

H₂N—R′—NH₂

wherein, each R′ is independently the same or different, substituted or unsubstituted divalent organic group, such as C₆₋₂₀ aromatic hydrocarbon group or halogenated derivative thereof, a straight or branched chain alkylene group or the halogen derivative thereof, a C₃₋₉ cycloalkylene group or halogen derivative thereof, in particular a divalent group of one or more of the following formulae:

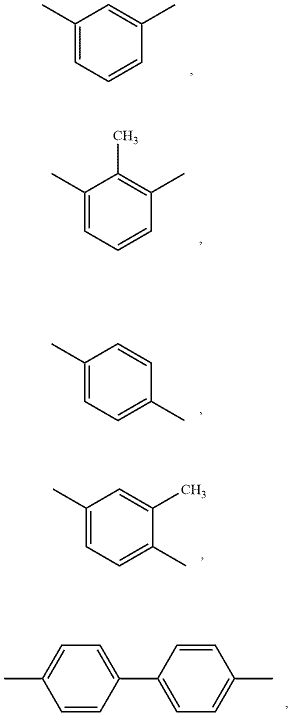

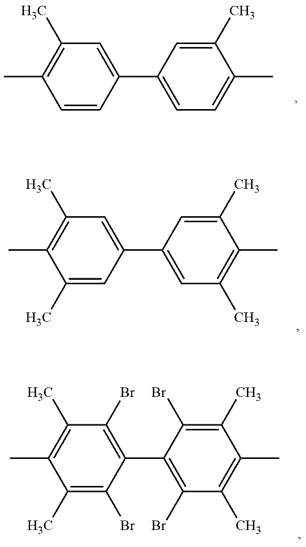

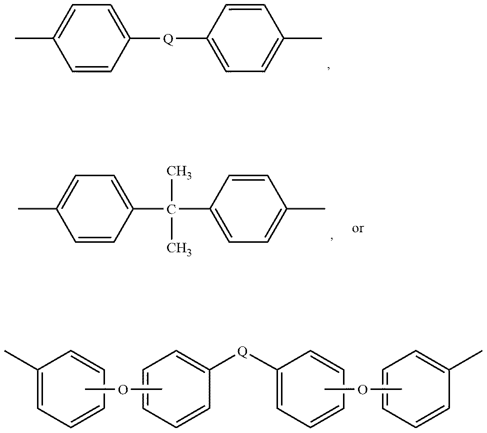

wherein Q is —O—, —S—, —C(O)—, —SO₂—, —SO—, —P(T)(═O)— wherein T is a C₁₋₈ alkyl or aryl, —C_{y}H_{2y}—wherein y is an integer from 1 to 5 or a halogenated derivative thereof (which includes perfluoroalkylene groups), or —(C₆H₁₀)_{z}— wherein z is an integer from 1 to 4.

In some aspects of the present invention R′ is m-phenylene, p-phenylene or a diarylene sulfone, in particular bis(4,4′-phenylene)sulfone, bis(3,4-phenylene) sulfone, bis(3,3′-phenylene) sulfone or combination including at least one of the foregoing.

Examples of organic diamines include ethylenediamine, propylenediamine, trimethylenediamine, diethylenediamine, triethylenetetramine, hexamethylenediamine, heptamethylenediamine, octamethylenediamine, nonamethylenediamine, decamethylenediamine, decamethylenediamine, 1,12-dodecamethylenediamine, 1,18-octadecamethylenediamine, 3-methylheptamethylenediamine, 4,4-dimethylpentamethylenediamine, 4-methylnanomethylenediamine, 5-methylnanomethylenediamine, 2,5-dimethylheptamethylenediamine, 2,2-dimethylpropylenediamine, N-methyl-bis(3-aminopropyl) amine, 3-methoxyhexamethylenediamine, 1,2-bis(3-aminopropoxy) ethane, bis(3-aminopropyl) sulfide, 1,4-cyclohexanediamine, bis(4-aminocyclohexyl) methane, m-phenylenediamine, p-phenylenediamine, 2,4-diaminotoluene, 2,6-diaminotoluene, m-xylenediamine, p-xylenediamine, 2-methyl-4,6-diethyl-1,3-phenylenediamine, 5-methyl-4,6-diethyl-1,3-phenylenediamine, benzidine, 3,3′-dimethylbenzidine, 3,3′-dimethoxybenzidine, 1,5-diaminonnaphthalene, bis(4-aminophenyl) methane, bis(2-chloro-4-amino-3,5-diethylphenyl)methane, bis(4-aminophenyl) propane, 2,4-bis(p-amino-t-butyl) toluene, bis(p-amino-t-butylphenyl) ether, bis(p-methyl-o-aminophenyl)benzene, bis(p-methyl-o-aminopentyl) benzene, 1,3-diamino-4-isopropylbenzene, bis(4-aminophenyl) sulfide, bis(4-aminophenyl) sulfone, and bis(4-aminophenyl) ether. Combination of these compounds can also be used.

In some aspects of the present invention the organic diamine is m-phenylenediamine, p-phenylenediamine, sulfonyldianiline, or a combination including one or more of the foregoing.

Copolymers of the polyimides can be manufactured using the combination of an aromatic dianhydride of the formula (12) and a different a dianhydride, for example a dianhydride wherein A does not contain an ether functionality, for example wherein A is a sulfone. Illustrative examples of dianhydride that can be prepared by the foregoing method or used to prepare polyimides include 3,3-bis[4-(3,4-dicarboxyphenoxy)phenyl]propane dianhydride, 4,4′-bis(3,4-dicarbophenoxy)diphenyl ether dianhydride, 4,4′-bis(3,4-dicarboxyphenoxy)diphenyl sulfide dianhydride, 4,4′-bis(3,4-dicarboxyphenoxy)benzophenone dianhydride, 4,4′-bis(3,4-dicarboxyphenoxy)diphenyl sulfide dianhydride, 4,4′bis-(3,4-dicarboxyphenoxy)diphenyl sulfone dianhydride, 2,2-bis[4-(2,3-dicarboxyphenoxy)phenyl]propane dianhydride, 4,4′-bis(2,3-dicarboxyphenoxy)diphenyl ether dianhydride, 4,4′-bis(2,3-dicarboxyphenoxy)diphenyl sulfide dianhydride, 4,4′-bis(2,3-dicarboxyphenoxy)benzophenone dianhydride, 4,4′-bis(2,3-dicarboxyphenoxy)diphenyl sulfone dianhydride, 4-(2,3-dicarboxyphenoxy)-4′-(3,4-dicarboxyphenoxy)diphenyl-2.2-propanedianhydride, 4-(2,3-dicarboxyphenoxy)-4′-(3,4-dicarboxyphenoxy)diphenyl ether dianhydride, 4-(2,3-dicarboxyphenoxy)-4′-(3,4-dicarboxyphenoxy)diphenyl sulfide dianhydride, 4-(2,3-dicarboxyphenoxy)-4′-(3,4-dicarboxyphenoxy)benzophenone dianhydride, and 4-(2,3-dicarboxyphenoxy)-4′-(3,4-dicarboxyphenoxy)diphenyl sulfone dianhydride, as well as various combinations thereof.

### Aspects and Embodiments of the Present Invention

A first aspect of the present invention includes a method of making a dianhydride which includes the reacting of nitro- or halo N-substituted phthalimide with bisphenol and sodium hydride in a polar aprotic solvents medium under condition to provide a reaction mixture including mono-imide and diimide, wherein the reaction temperature is between about 0 to about 250° C.; preferably removing most or all of the solvents by precipitating in acetic acid solution, filtering the precipitate; reacting the resulting diimide with a carboxylic acid and substituted or unsubstituted dimethyl sulfoxide in an aqueous medium under conditions to provide a reaction mixture including a tetra acid, a triacid and an imide diacid, wherein the reaction temperature is between about 160 to about 250° C. and reaction pressure is between about 150 to about 300 psig, preferably between about 170 to about 250 psig; removing the sulfoxide, carboxylic acids, and other byproducts by precipitation in water; filtering the precipitate ; and converting the tetra acid precipitate to the corresponding dianhydride; wherein
N-substituted phthalimide is of the formula

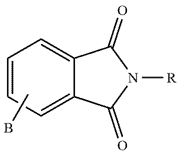

Bisphenol is of the formula

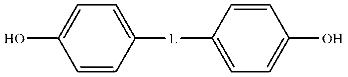

diimide is of the formula

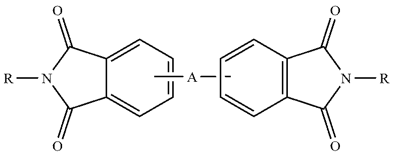

The carboxylic acid is of formula X—COOH
Sulfoxide is of formula J₂SO
tetra acid is of formula

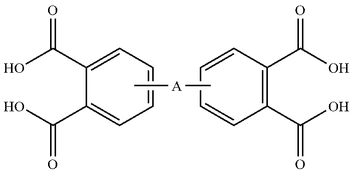

triacid is of formula

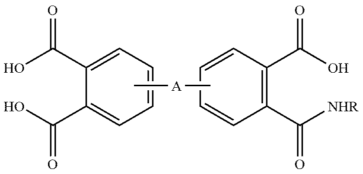

diacid imide is of formula

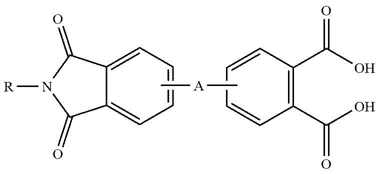

Dianhydride is of formula

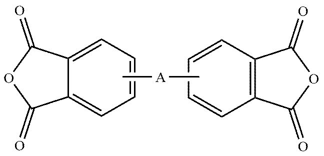

wherein in the forgoing formulas
B is —NO2, —Cl, —F, —Br, or —I.
A is —O—, —S—, —C(O)—, —SO₂—, —SO—, —C_{y}H_{2y}— wherein y is an integer from 1 to 5 or a halogenated derivative thereof or —O—E—O—, wherein E is an aromatic C₆₋₂₄ monocyclic or polycyclic moiety optionally substituted with 1 to 6 of C₁₋₈ alkyl groups, 1 to 8 halogen atoms, or a combination including at least one of the foregoing.
R is a monovalent C₁₋₁₃ organic group; X is aryl group, C₁₋₈ alkyl group, or preferably a methyl group.
J is C₁₋₈ alkyl group, or aryl group, preferably a methyl group.

Another aspect of the present invention includes wherein E is 2,2-(4-phenylene)isopropylidene.

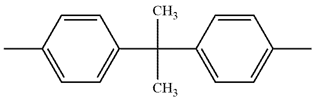

Another aspect of the present invention includes wherein E is 4-phenylene-1.1′-biphenyl.

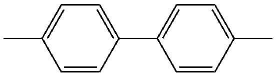

In an aspect of the present invention, the initial molar ratio of bisphenol: nitro or halo N-substituted phthalimide: to sodium hydride is between about 1:1.8:1.8 to about 1:2.2:2.2

In an aspect of the present invention, the initial mass ratio of bisphenol and dimethylsulfoxide is between about 1:2 to about 1:20

A further aspect of the present invention includes wherein the initial mass ratio of acetic acid to diimide is between about 1:1 to about 50:1, or about 1:1 to about 20:1, or about 1:1 to about 10:1.

An additional aspect of the present invention includes wherein the initial mass ratio of dimethyl sulfoxide to diimide is between about 1:1 to about 50:1, or about 1:1 to about 20:1, or about 1:1 to about 10:1.

An additional aspect of the present invention includes wherein the initial mass ratio of water to diimide is between about 1:1 to about 100:1, or about 2:1 to about 50:1, or about 2:1 to about 20:1.

Another aspect of the present invention includes wherein the reaction mixture further includes the diimide, acetic acid with its derivatives, and dimethyl sulfoxide and its reaction and decomposition products.

A further aspect of the present invention includes wherein the precipitation is done by adding into water.

An additional aspect of the present invention includes wherein the precipitation is done by cooling the reaction mixture to between about 5 to about 50° C.

Another aspect of the present invention includes wherein the ratio of reaction mixture to water for precipitation is between about 1:0 to about 1:10.

A further aspect of the present invention includes wherein the precipitate is heated at 180 to 250° C. under the reduced pressure of less than about 200 mm/Hg with or without a dehydrating agent.

An additional aspect of the present invention includes wherein the reaction mixture is directly converted into dianhydride by heating at between about 180 to about 250° C. under the reduced pressure less than about 200 mm of Hg with or without the dehydrating agent.

Another aspect of the present invention includes wherein conversion of diimide to dianhydride is at least about 90%, preferably at least about 96%.

An additional aspect of the present invention includes wherein the diimide includes 4,4′-bisphenol A-bis-N-methylphthalimide, 3.4′-bisphenol A-bis-N-methylphthalimide, 3,3′-bisphenol A-bis-N-methylphthalimide, or a combination including at least one of the foregoing; the dianhydride includes 4,4′-bisphenol A-bis-dianhydride, 3,4′-bisphenol A-bisdianhydride, 3,3′-bisphenol A-bis-dianhydride, or a combination including at least one of the forgoing.

Another aspect of the present invention includes wherein imide anhydride is present in an amount of less than about 10%, preferably less than about 4%, based on the total weight of the imide anhydride and dianhydride.

A further aspect of the present invention includes wherein the product dianhydride contains traces of diimide.

An additional aspect of the present invention includes wherein the dianhydride contains the dimethyl sulfoxide and its derivatives as impurities.

Another aspect of the present invention includes wherein the dianhydride contains the acetic acid and its derivatives as impurities.

A further aspect of the present invention includes a method for manufacture of polyimide composition, the method including manufacturing a dianhydride in accordance with a method of any or more of the proceeding claims; polymerizing the dianhydride and a diamine to provide a polyetherimide composition.

This disclosure is further illustrated by the examples, which are not limiting.

### II A Dianhydride Made by a Method of the Present Invention

The present invention also includes a diimide and dianhydride made by a method of the present invention.

The present invention generally includes a diimide made by the method of the present invention, wherein the diimide contains a trace of monoimide.

The present invention generally includes a dianhydride made by a method of the present invention, wherein the dianhydride has an imide anhydride content of about 0.1 to about 10% based on the total weight of the aromatic dianhydride.

The present invention generally includes a dianhydride made by a method of the present invention, wherein the dianhydride contains traces of diimide.

The present invention generally includes a dianhydride made by a method of the present invention, wherein the dianhydride contains traces of dimethyl sulfoxide and their derivatives as impurities.

The present invention generally includes a dianhydride made by a method of the present invention, wherein the dianhydride contains traces of acetic acid and their derivatives as impurities.

Another aspect of the present invention includes a polyetherimide composition manufactured by a method of the present invention.

This disclosure is further illustrated by the examples, which are not limiting.

### III Additional Aspects and Embodiments of the Present Invention

Further included in this disclosure are the following specific aspects of the present invention, which do not limit the claims.

Aspect 1: A method for manufacture of diimide, the method including contacting the bisphenol with sodium hydride and halo or nitro N-substituted phthalimide under the conditions effective to provide a composition including diimide.

Aspect 2: The method of Aspect 1, wherein contacting the bisphenol with sodium hydride and halo or nitro N-substituted phthalimide is conducted in presence of polar aprotic solvents.

Aspect 3: The method of Aspect 1 to 2, wherein polar aprotic solvent is dimethyl sulfoxide, sulfolane, dimethylacetamide, dimethylformamide, tetrahydrofuran, acetonitrile, N-methyl-2-pyrilodone (NMP), O-cresol, P-cresol or a combination including at leat one of the foregoing.

Aspect 4: The method of Aspect 1 to 2, wherein N-substituted phthalimide is chloro N-methylphthalimide, nitro N-methylphthalimide, or a combination including at least one of the foregoing.

Aspect 5: The method of Aspect 1 to 2, wherein biphenol is Bisphenol A, Biphenol, hydroquinone, resorcinol, dihydroxydiphenyl sulfone, or a combination including at least one of the foregoing.

Aspect 6: The method of Aspect 1 to 5, wherein the molar ratio of bisphenol to halo or nitro N-substituted phthalimide is between about 1.0:1.8 to about 1.0:2.2.

Aspect 7: The method of Aspect 1 to 6, wherein the molar ratio of bisphenol to sodium hydride is between about 1.0:1.8 to about 1.0:2.2.

Aspect 8: The method of Aspect 1 to 7, wherein the mass ratio of bisphenol to polar aprotic solvent is between about 1:2 to about 1:20.

Aspect 9: The method of any one or more of the proceeding Aspects, wherein contacting the bisphenol with sodium hydride and halo or nitro N-substituted phthalimide with polar aprotic solvent is conducted at a temperature of between about 0 to about 250° C.

Aspect 10: The method of any one or more of the proceeding Aspects, wherein the reaction mixture is precipitated in acetic acid solution

Aspect 11: The method of any one or more of the proceeding Aspects, wherein the halo or nitro N-substituted phthalimide is of the formula,

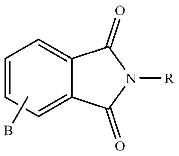

bisphenol of the formula

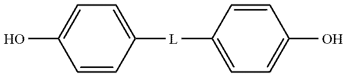

diimide of the formula

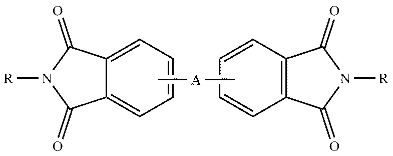

Aspect 12: A method for the manufacture of dianhydride, the method including contacting a N-substituted diimide with an organic sulfoxide and carboxylic acid under conditions effective to provide a composition including the dianhydride.

Aspect 13: The method of Aspect 12, wherein contacting the N-substituted diimide with organic sulfoxide and carboxylic acid is conducted in the presence of water.

Aspect 14: The method of Aspect 12 to 13, wherein the organic sulfoxide is substituted or unsubstituted dimethyl sulfoxide, dialkyl sulfoxide, diaryl sulfoxide, or a combination including at least one of the foregoing.

Aspect 15: The method of Aspect 12 to 13, wherein carboxylic acid is substituted or unsubstituted acetic acid, aryl carboxylic acid, or combination including at least one of the foregoing.

Aspect 16: The method of Aspect 12 to 15, wherein the mass ratio of organic sulfoxide relative to N-substituted diimide is about 1:1 to about 10:1.

Aspect 17: The method of Aspect 12 to 16, wherein the mass ratio of carboxylic acid relative to N-substituted diimide is between about 1:1 to about 10:1.

Aspect 18: The method of Aspect 12 to 17, wherein the mass ratio of water relative to N-substituted diimide is between about 2:1 to about 20:1.

Aspect 19: The method of any one or more of the proceeding Aspects, wherein contacting the N-substituted diimide with organic sulfoxide and carboxylic acid in aqueous medium is conducted at a temperature of between about 150 to about 230° C.

Aspect 20: The method of any one or more of the proceeding Aspects, wherein contacting the N-substituted diimide with organic sulfoxide and carboxylic acid is conducted at a pressure of between about 150 to about 250 psi.

Aspect 21: The method of any one or more of the proceeding Aspects, wherein the reaction mixture is precipitated in water or by itself on cooling.

Aspect 22: The method of any one or more of the proceeding Aspects, wherein heating the precipitation with tetra acid provides a composition including the dianhydride.

Aspect 23: The method of any one or more of the proceeding Aspects, wherein heating the reaction mixture with tetra acid provides a composition including the dianhydride.

Aspect 24: The method of any one or more of the proceeding Aspects, wherein heating the reaction mixture with tetra acid is carried out at the temperature of between about 140 to about 220° C.

Aspect 25: The method of any one or more of the proceeding Aspects, wherein heating the reaction mixture with tetra acid is carried out at the pressure of between about 200 mm of Hg or less.

Aspect 26: The method of any one or more of the proceeding Aspects, wherein the N-substituted diimide is of the formula

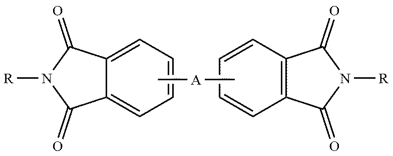

the tetra acid of the formula

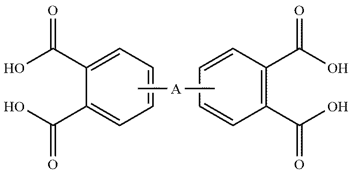

the triacid of the formula

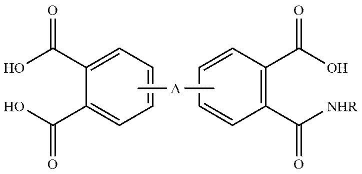

imide diacid is of formula

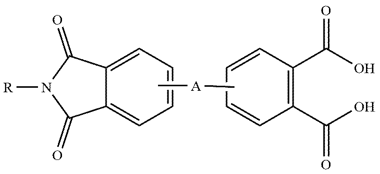

the dianhydride is of the formula

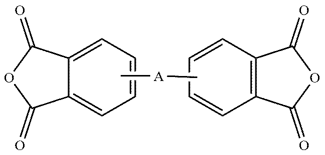

wherein, in the foregoing formulas, B is —NO2, —Cl, —F, —Br, or —I; R is an aryl, a C₁₋₅ alkyl, preferable methyl; and A is —O—, or a group of formula —O—E—O—, wherein E is of the formula

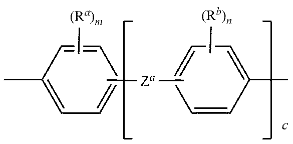

wherein R^{a} and R^{b} are each independently a halogen atom or a monovalent C₁₋₆ alkyl group and can be the same or different; m and n are each independent integers of 0 to 4; c is 0 to 4, specifically 0 or 1; and Z^{a} is a bridging group connecting the two aromatic groups, where the bridging group and point of attachment of each C₆ arylene group are disposed ortho, meta, or para (specifically para) to each other on the C₆ arylene group. The bridging group Z^{a} can be a single bond, —O—, —S—, —S(O)—, —S(O)₂—, —C(O)—, or a C₁₋₁₈ organic bridging group. The C₁₋₁₈ organic bridging group can be cyclic or acyclic, aromatic or non-aromatic, and can further include heteroatoms such as halogens, oxygen, nitrogen, sulfur, silicon, or phosphorus. The C₁₋₁₈ organic group can be disposed such that the C₆ arylene groups connected thereto are each connected to a common alkylidene carbon or to different carbons of the C₁₋₁₈ organic bridging group. A specific example of a group E is a divalent group of formula

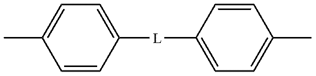

wherein L is a single bond, —O—, —S—, —C(O)—, —SO₂—, —SO—, —C_{y}H_{2y}— and a halogenated derivative thereof wherein y is an integer from 1 to 5.

Aspect 27: The method of Aspect 26, wherein E is 2,2(4-phenylene)isopropylidene of formula

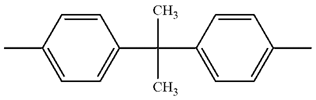

Aspect 28: The method of Aspect 26, wherein E is is also 4-phenylene-1.1′-biphenyl of formula

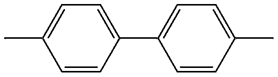

Aspect 29: A method for the manufacture of a polyetherimide composition, the method including manufacturing dianhydride in accordance with a method of any or more of the proceeding Aspects; polymerizing the dianhydride and a diamine to provide a polyimide composition.

Aspect 30: A polyetherimide composition manufactured by the method of Aspect 29.

### IV Preferred Aspects and Embodiments of the Present Invention

Further included in this disclosure are the following specific aspects of the present invention, which do not limit the claims.

A first preferred aspect of the present invention includes a method of making a dianhydride, which includes:
a) combining bisphenol with halo or nitro N-substitued phthalimide and sodium hydride in a polar aprotic solvent to provide at least one mono-imide, at least one diimide, or a combination thereof in a solution;
b) isolating said diimide from said solution to form an isolate;
c) combining said isolated diimide with carboxylic acid and dimethylsulfoxide to provide at least one tetra acid, at least one triacid, at least one imide diacid, or a combination thereof;
d) precipitating said tetra acid, said triacid, said imide diacid, or a combination thereof to form a precipitate;
e) removing the sulfoxide, carboxylic acids, and other byproducts from said precipitate; and
f) converting said precipitate to the corresponding dianhydride;
   wherein said halo and nitro N-substituted phthalimide is of formula 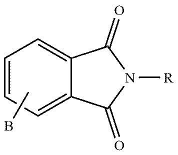
   wherein said bisphenol is of formula 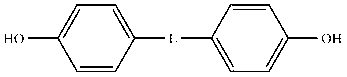
   wherein said diimide is of the formula 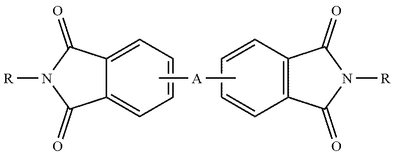
   wherein said carboxylic acid is of formula X—COOH
   wherein said sulfoxide is of formula J₂SO
   wherein said tetra acid is of formula, 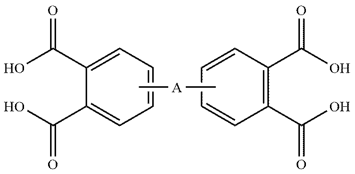
   wherein said triacid is of formula, 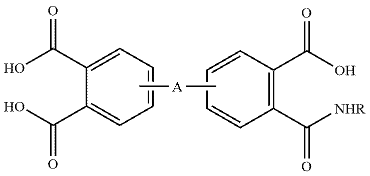
   wherein said diacid imide is of the formula, 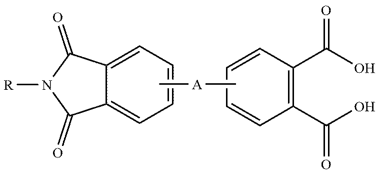
   wherein said dianhydride is of the formula, 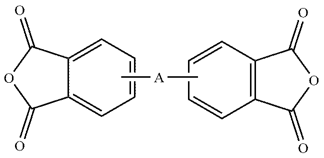
   wherein in the forgoing formulas
      B is —NO2, —Cl, —F, —Br, or —I;
      A is —O—, —S—, —C(O)—, —SO₂—, —SO—, -C_{y}H_{2y}-
         wherein y is an integer from 1 to 5 or
         a halogenated derivative thereof or —O—E—O—,
         wherein E is an aromatic C₆₋₂₄ monocyclic or polycyclic moiety optionally substituted with 1 to 6 of C₁₋₈ alkyl groups, 1 to 8 halogen atoms, or a combination comprising at least one of the foregoing;
   wherein R is a monovalent C₁₋₁₃ organic group;
   wherein X is aryl group, C₁₋₈ alkyl group, or preferably a methyl group.
   wherein J is C₁₋₈ alkyl group, or aryl group, preferably a methyl group.

In an aspect of the present invention; the method includes the reacting a halo- or nitro- N-substituted phthalimide with bisphenol and sodium hydride in polar aprotic solvent under condition to provide diimide which on reacting with a carboxylic acid and substituted or unsubstituted dimethyl sulfoxide in an aqueous medium under conditions to provide a reaction mixture.

In a further aspect of the present invention; in a) the reaction temperature is between about 0 to about 250° C.

In an additional aspect of the present invention; in b) the isolating is by precipitating in acetic acid solution.

In an aspect of the present invention; in c) the reaction temperature is between about 160 to about 250° C.

In a further aspect of the present invention; in c) the reaction pressure is between about 150 to about 300 psig, between about 170 to about 250 psig, or a combination thereof.

In an additional aspect of the present invention; in d) the precipitating is performed in water.

In an aspect of the present invention; in e) the removing is performed by filtering the precipitate.

In a further aspect of the present invention; E is 2,2-(4-phenylene)isopropylidene.

In an additional aspect of the present invention; E is 4-phenylene-1,1′-biphenyl.

In an aspect of the present invention; in c) the initial mass ratio of acetic acid to diimide is between about 1:1 to about 50:1, or about 1:1 to about 20:1, or about 1:1 to about 10:1.

In a further aspect of the present invention; in c) the initial mass ratio of dimethyl sulfoxide to diimide is between about 1:1 to about 50:1, or about 1:1 to about 20:1, or about 1:1 to about 10:1.

In an aspect of the present invention; in c) the initial mass ratio of water to diimide is between about 1:1 to about 100:1, or about 2:1 to about 50:1, or about 2:1 to about 20:1.

In an additional aspect of the present invention; in c) the reaction mixture further comprises the diimide, acetic acid with its derivatives, and dimethyl sulfoxide and its reaction and decomposition products.

In an aspect of the present invention; in d) the precipitation is done by adding into water.

In a further aspect of the present invention; in d) the precipitation is done by cooling the reaction mixture to between about 5 to about 50° C.

In an additional aspect of the present invention; in d) the ratio of reaction mixture to water for precipitation is between about 1:0 to about 1:10.

In an aspect of the present invention; in 1 f) the precipitate is heated at 180 to 250° C. under the reduced pressure of less than about 200 mm/Hg.

In a further aspect of the present invention; in f) the reaction mixture is directly converted into dianhydride by heating at between about 180 to about 250° C. under the reduced pressure less than about 200 mm of Hg.

In an additional aspect of the present invention; the conversion of diimide to dianhydride is at least about 90%, preferably at least about 96%.

In a further additional aspect of the present invention; the diimide comprises 4,4′-bisphenol A-bis-N-methylphthalimide, 3.4′-bisphenol A-bis-N-methylphthalimide, 3,3′-bisphenol A-bis-N-methylphthalimide, or a combination comprising at least one of the foregoing; the diimide also comprises 4,4′-biphenol-N-methylphthalimide, 3,4′-biphenol-N-methylphthalimide, 3,3′-biphenol-N-methylphthalimide or a combination comprising at least one of the foregoing; the dianhydride comprises 4,4′-bisphenol A-bis-dianhydride, 3,4′-bisphenol A-bisdianhydride, 3,3′-bisphenol A-bis-dianhydride, or a combination comprising at least one of the forgoing; the dianhydride also comprises 4,4′-biphenol-bisanhydride, 3,4′-biphenol-bisanhydride, 3,3′-biphenol-bisanhydride or a combination comprising at least one of the forgoing.

In an aspect of the present invention; imide anhydride is present in an amount of less than about 10%, preferably less than about 4%, based on the total weight of the imide anhydride and dianhydride.

In a further aspect of the present invention; the product dianhydride contains traces of diimide.

In an additional aspect of the present invention; the dianhydride contains the dimethyl sulfoxide and its derivatives as impurities.

In an aspect of the present invention; the dianhydride contains the acetic acid and its derivatives as impurities.

A second preferred aspect of the present invention includes a method for manufacture of polyetherimide composition, the method including:
a) manufacturing a dianhydride in accordance with any method of a method of method of making a dianhydride of the present invention.
b) polymerizing the dianhydride and a diamine to provide a polyetherimide composition.

A third preferred aspect of the present invention includes a polyetherimide composition manufactured by any method of manufacture of polyetheriide composition of the present invention.

The compositions, methods, and articles can alternatively include, consists of, or consists essentially of, any appropriate materials, or components herein disclosed. The compositions, methods, and articles can additionally, or alternately, be formulated so as to be devoid, or substantially free, of any materials (or species), steps, or components, that are otherwise not necessary to the achievement of the function or objective of the compositions, methods, and articles.

### EXAMPLES

### Example 1: Synthesis of the Starting Material Diimide

This example establishes the preparation of the starting material diimide using the previously established method.

Bisphenol A diimide (3):

Bisphenol A diimide was prepared based on the procedure described in U.S. Pat. No: 3,879,428. A 250 ml three-neck round bottomed flask containing a magnetic stirrer bar was fitted with a thermocouple, a nitrogen inlet and a nitrogen outlet with a bubbler through a Dean-Stark trap. The flask was charged with NaOH (1.6 g, 0.04 moles, 2.00 equivalents) and water (1.6 ml). The flask was placed in a heating mantle and stirred at room temperature until a solution was formed. Bisphenol A (4.566 g, 0.02 moles, 1.00 equivalents), toluene (50 ml) and DMSO (30 ml) were added to that solution. The temperature of the heating mantle was slowly increased to 85° C. and continued to stir while distilling off the azeotropic mixture of toluene and water. The distilling was continued for 3 hours while all the toluene and water removed from the reaction mixture. The temperature of the system was slowly increased to 160° C. and heated for another hour. To the mixture, N-methyl-4-nitrophthalimide (8.6587 g, 0.042 moles, 2.10 equivalents) was added as solid and heating was continued for another four hours. HPLC analysis of the reaction mixture showed the consumption of most of the starting material N-methylphthalimide. The reaction flask was cooled to 70° C. and filtered through a 90 mm filter paper using suction filtration set up to remove the salt. The filtrate dark solution was slowly poured into 200 ml water while stirring with a spatula to precipitate the product bisphenol A diimide. The product slurry in water was filtered through 90 mm wet strengthened filter paper with suction apparatus. The precipitate was washed with 50 ml water two more times and dried overnight at 100° C. under vacuum to obtain 8.7 g of the product. The dark color of the product was removed by dissolving in methylene chloride and filtering through a silica plug.

### Example 2: Synthesis of the Starting Material Diimide Using Sodium Hydride as a Base

This example establishes the preparation of the starting material diimide using a newly invented method.

Bisphenol A diimide (3):

A 250 ml three-neck round bottomed flask containing a magnetic stirrer bar was fitted with a thermocouple, a nitrogen inlet and a nitrogen outlet with a bubbler through a Dean-Stark trap. The flask was charged with anhydrous dimethyl sulfoxide and Bisphenol A (4.566 g, 0.02 moles, 1.00 equivalents). The flask was placed in a cold-water bath and until a solution was formed. To this solution, sodium hydride (1.6 g, 0.04 moles, 2.0 equivalents, a 60 % suspension solid in mineral oil) was added as solid in portions and continued stirring for 1 h. To the mixture, N-methyl-4-nitrophthalimide (8.251 g, 0.04 moles, 2.00 equivalents) was added as solid and the temperature of the stirring mixture was slowly increased to 160° C. and stirring continued for four hours. HPLC analysis of the reaction mixture showed the consumption of most of the starting material N-methylphthalimide. The reaction flask was cooled to 70° C. and filtered through a 90 mm filter paper using suction filtration set up to remove the salt. The filtrate dark solution was slowly poured into 200 ml acetic acid solution while stirring with a spatula to precipitate the product bisphenol A diimide. The product slurry in water was filtered through 90 mm wet strengthened filter paper with suction apparatus. The precipitate was washed with 50 ml water two more times and dried overnight at 100° C. under vacuum to obtain 8.2 g of the product. The brown color of the product was removed by dissolving in methylene chloride and filtering through a silica plug.

### Example 3: Synthesis of Dianhydride From the Starting Material Diimide

This example establishes that the preparation of the dianhydride from the starting material diimide is possible in high yield if the diimide is reacted with acetic acid and dimethyl sulfoxide in aqueous medium at high temperature and high pressure followed by ring closing of the resulting tetra acids.

In an autoclave reactor with a magnetic stirrer bar, bisphenol A diimide (4.372 g, 0.008 moles, 1.00 equivalent), dimethyl sulfoxide (9.37 g, 0.12 moles, 15.00 equivalents), acetic acid (9.6 g, 0.16 moles, 20.00 equivalents), and 15 ml water were placed. The reactor was heated at 190° C. and pressure of 200 psi while stirring for 6 hours. The reactor was cooled to room temperature. LCMS of the reaction mixture showed the exclusive conversion of starting diimide (3) into the tetra acid with traces of the starting material and partial hydrolyzed product left.

The reaction mixture was diluted with water (30 ml) to precipitate the resulting tetra acid. The aqueous slurry was centrifuged to obtain a solid which was dried under vacuum at room temperature.

The solid tetra acid (2.0 g) was flushed with nitrogen and heated to 200° C. under vacuum for 2 h and cooled to room temperature. LCMS analysis of the resulting solid showed the formation of dianhydride of the formula (13),

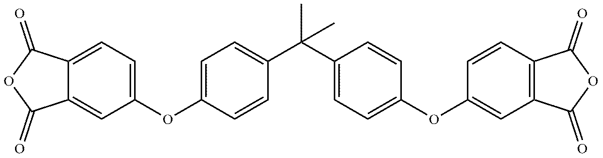 [SMILES: CC(C)(c1ccc(Oc2ccc3c(c2)C(=O)OC3=O)cc1)c1ccc(Oc2ccc3c(c2)C(=O)OC3=O)cc1]

### Example 4: Making Dianhydride in the Absence of Acetic Acid

This example establishes that synthesis of dianhydrides from diimides is either difficult or not possible or very low yielding if acetic acid is not present in the method described in the Example 3.

In an autoclave reactor with a magnetic stirrer bar, bisphenol A diimide (4.372 g, 0.008 moles, 1.00 equivalent), dimethyl sulfoxide (9.37 g, 0.12 moles, 15.00 equivalents), and 13 ml water were placed. The reactor was heated at 190° C. at 200 psi while stirring for 6 hours. The reactor was cooled to room temperature. LCMS of the reaction mixture showed no noticeable conversion of the starting material.

### Example 5: Making the Dianhydride in the Absence of Dimethyl Sulfoxide

This example establishes that synthesis of dianhydrides from diimides is either difficult or not possible or very low yielding if dimethyl sulfoxide is not present in the method described in the Example 3.

In an autoclave reactor with a magnetic stirrer, bisphenol A diimide (4.372 g, 0.008 moles, 1.00 equivalent), acetic acid (9.6 g, 0.16 moles, 20.00 equivalents), and 13 ml water were placed. The reactor was heated at 190° C. at 200 psi while stirring for 6 hours. The reactor was cooled to room temperature. LCMS of the reaction mixture showed no noticeable conversion of the starting material.

### Example 6: Making the Dianhydride in the Absence of Water

This example establishes that synthesis of dianhydrides from diimides is either difficult or not possible or very low yielding if water is not present in the method described in the Example 3.

In an autoclave reactor with a magnetic stirrer, bisphenol A diimide (4.372 g, 0.008 moles, 1.00 equivalent), dimethyl sulfoxide (9.37 g, 0.12 moles, 15.00 equivalents), and acetic acid (9.6 g, 0.16 moles, 20.00 equivalents) were placed. The reactor was heated at 190° C. at 200 psi while stirring for 6 hours. The reactor was cooled to room temperature. LCMS of the reaction mixture showed no noticeable conversion of the starting material.

### Example 7: Synthesis of Dianhydride From N-methyl Nitrophthalimide And Biphenol

A 250 ml three-neck round bottomed flask containing a magnetic stirrer bar was fitted with a thermocouple, a nitrogen inlet and a nitrogen outlet with a bubbler through a Dean-Stark trap. The flask was charged with biphenol (3.72 g, 0.02 moles, 1.00 equivalents) and DMSO (50 ml). To this stirring solution, sodium hydroxide (1.76 g, 0.042 mol, 2.10 equivalent) added as 50% solution in water. The reaction flask was heated to 90° C. for 2 h in oil bath. 20 ml toluene was added to this mixture and the water-toluene was azeotroped into the Dean-Stark trap. To the stirring dry reaction mixture at 100° C., N-methyl-4-nitrophthalimide (8.658 g, 0.042 moles, 2.10 equivalents) was added and the stirring continued for 2 hours. LCMS analysis of the reaction mixture showed the consumption of most of the starting materials and formation of the biphenol diimide as the major product. The reaction mixture was poured in 5% acetic acid solution in 200 ml water. The precipitate was stirred for 15 min and the solid diimide (9.2 g, 91%) was recovered by filtration.

The solid diimide (1.0 g) was transferred into a 50 ml autoclave reactor with a magnetic stirrer bar. DMSO (2 ml), acetic acid (2 ml) and water (8 ml) were added. The reactor was sealed and heated at 190° C. and pressure of 200 psi overnight while stirring. The reactor was cooled to room temperature. LCMS of the reaction mixture showed the exclusive conversion of diimide into the tetra acid with traces of the starting material and partial hydrolyzed product left.

The reaction mixture was diluted with water (10 ml) to precipitate the resulting tetra acid. The aqueous slurry was centrifuged to obtain a solid which was dried under vacuum at room temperature.

The solid tetra acid (1.0 g) was flushed with nitrogen and heated to 200° C. under vacuum for 2 h and cooled to room temperature. LCMS analysis of the resulting solid (0.8 g) showed the formation of dianhydride of the formula (14),

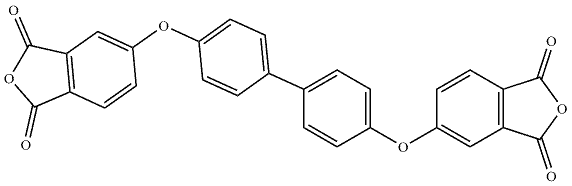 [SMILES: O=C1OC(=O)c2cc(Oc3ccc(-c4ccc(Oc5ccc6c(c5)C(=O)OC6=O)cc4)cc3)ccc21]

### Example 8: Synthesis of Dianhydride From N-methyl Nitrophthalimide and Hydroquinone

A 250 ml three-neck round bottomed flask containing a magnetic stirrer bar is fitted with a thermocouple, a nitrogen inlet and a nitrogen outlet with a bubbler through a Dean-Stark trap. The flask is charged with hydroquinone (0.55 g, 0.005 moles, 1.00 equivalents) and DMSO (10 ml). To this stirring solution, sodium hydroxide (0.42 g, 0.0105 mol, 2.1 equivalent) added as 50% solution in water. The reaction flask is heated to 90° C. in oil bath until the salt formation is complete. 20 ml toluene is added to this mixture and the water toluene is azeotroped into the Dean-Stark trap. To the stirring dry reaction mixture at 100° C., N-methyl-4-nitrophthalimide (2.164 g, 0.0105 moles, 2.10 equivalents) is added and the stirring continued until the reaction is complete. Once complete, the DMSO solution of the diimide reaction mixture is poured into 5% acetic acid solution to precipitate the diimide as solid.

The resulting diimide solid is transferred into a 50 ml autoclave reactor with a magnetic stirrer bar. DMSO (5 ml) acetic acid (5 ml) and water (15 ml) are added to the solution. The reactor is sealed and heated at 190° C. and pressure of 200 psi while stirring. Once complete the reactor is cooled to room temperature. The reaction mixture is diluted with water (30 ml) to precipitate the resulting tetra acid. The aqueous slurry is centrifuged to obtain a solid which is dried under vacuum at room temperature.

The solid tetra acid is flushed with nitrogen and heated to 200° C. under vacuum to obtain the dianhydride of the formula (15),

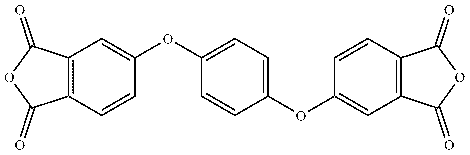 [SMILES: O=C1OC(=O)c2cc(Oc3ccc(Oc4ccc5c(c4)C(=O)OC5=O)cc3)ccc21]

### Example 9: Synthesis of Dianhydride From N-methyl Nitrophthalimide and Bisphenol A

A 250 ml three-neck round bottomed flask containing a magnetic stirrer bar is fitted with a thermocouple, a nitrogen inlet and a nitrogen outlet with a bubbler through a Dean-Stark trap. The flask is charged with bisphenol A (2.164 g, 0.005 moles, 1.00 equivalents) and DMSO (10 ml). To this stirring solution, sodium hydroxide (0.42 g, 0.0105 mol, 2.1 equivalent) added as 50% solution in water. The reaction flask is heated to 90° C. in oil bath until the salt formation is complete. 20 ml toluene is added to this mixture and the water toluene is azeotroped into the Dean-Stark trap. To the stirring dry reaction mixture at 100° C., N-methyl-4-nitrophthalimide (2.164 g, 0.0105 moles, 2.10 equivalents) is added and the stirring continued until the reaction is complete. Once complete, the DMSO solution of the diimide reaction mixture is poured into 5% acetic acid solution to precipitate the diimide as solid.

The resulting diimide solid is transferred into a 50 ml autoclave reactor with a magnetic stirrer bar. DMSO (5 ml) acetic acid (5 g) and water (15 ml) are added to the solution. The reactor is sealed and heated at 190° C. and pressure of 200 psi while stirring. Once complete the reactor is cooled to room temperature. The reaction mixture is diluted with water (30 ml) to precipitate the resulting tetra acid. The aqueous slurry is centrifuged to obtain a solid which is dried under vacuum at room temperature.

The solid tetra acid is flushed with nitrogen and heated to 200° C. under vacuum to obtain the dianhydride of the formula (13),

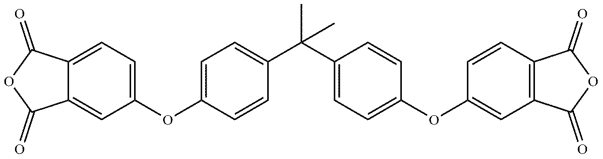 [SMILES: CC(C)(c1ccc(Oc2ccc3c(c2)C(=O)OC3=O)cc1)c1ccc(Oc2ccc3c(c2)C(=O)OC3=O)cc1]

### Example 10: Synthesis of Dianhydride From N-methyl Nitrophthalimide and 4,4′-Dihydroxydiphenyl Sulfone

A 250 ml three-neck round bottomed flask containing a magnetic stirrer bar iss fitted with a thermocouple, a nitrogen inlet and a nitrogen outlet with a bubbler through a Dean-Stark trap. The flask is charged with 4,4′-dihydroxydiphenyl sulfone (1.251 g, 0.005 moles, 1.00 equivalents) and DMSO (50 ml). To this stirring solution, sodium hydroxide (0.440 g, 0.0105 mol, 2.10 equivalent) added as 50% solution in water. The reaction flask is heated to 90° C. in oil bath until the salt formation is complete. 20 ml toluene is added to this mixture and the water toluene is azeotroped into the Dean-Stark trap. To the stirring dry reaction mixture at 100° C., N-methyl-4-nitrophthalimide (2.165 g, 0.0105 moles, 2.10 equivalents) is added and the stirring continued until the reaction is complete. Once complete, the DMSO solution of the diimide reaction mixture is poured into 5% acetic acid solution to precipitate the diimide as solid.

The resulting diimide solid is transferred into a 50 ml autoclave reactor with a magnetic stirrer bar. DMSO (5 ml) acetic acid (5 g) and water (15 ml) are added to the solution. The reactor is sealed and heated at 190° C. and pressure of 200 psi while stirring. Once complete the reactor is cooled to room temperature. The reaction mixture is diluted with water (30 ml) to precipitate the resulting tetra acid. The aqueous slurry is centrifuged to obtain a solid which is dried under vacuum at room temperature.

The solid tetra acid is flushed with nitrogen and heated to 200° C. under vacuum to obtain the dianhydride of the formula (16),

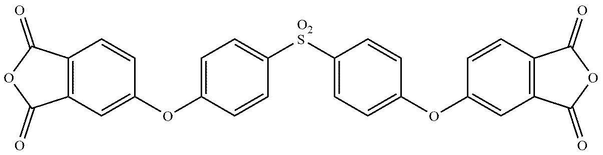 [SMILES: O=C1OC(=O)c2cc(Oc3ccc(S(=O)(=O)c4ccc(Oc5ccc6c(c5)C(=O)OC6=O)cc4)cc3)ccc21]

### Example 11: Synthesis of Dianhydride From N-methyl Nitrophthalimide and Resorcinol

A 250 ml three-neck round bottomed flask containing a magnetic stirrer bar iss fitted with a thermocouple, a nitrogen inlet and a nitrogen outlet with a bubbler through a Dean-Stark trap. The flask is charged with resorcinol (0.550 g, 0.005 moles, 1.00 equivalents) and DMSO (50 ml). To this stirring solution, sodium hydroxide (0.420 g, 0.0105 mol, 2.10 equivalent) added as 50% solution in water. The reaction flask is heated to 90° C. in oil bath until the salt formation is complete. 20 ml toluene is added to this mixture and the water toluene is azeotroped into the Dean-Stark trap. To the stirring dry reaction mixture at 100° C., N-methyl-4-nitrophthalimide (2.165 g, 0.105 moles, 2.10 equivalents) is added and the stirring continued until the reaction is complete. Once complete, the DMSO solution of the diimide reaction mixture is poured into 5% acetic acid solution to precipitate the diimide as solid.

The resulting diimide solid is transferred into a 50 ml autoclave reactor with a magnetic stirrer bar. DMSO (5 ml) acetic acid (5 g) and water (15 ml) are added to the solution. The reactor is sealed and heated at 190° C. and pressure of 200 psi while stirring. Once complete the reactor is cooled to room temperature. The reaction mixture is diluted with water (30 ml) to precipitate the resulting tetra acid. The aqueous slurry is centrifuged to obtain a solid which is dried under vacuum at room temperature.

The solid tetra acid is flushed with nitrogen and heated to 200° C. under vacuum to obtain the dianhydride of the formula (17),

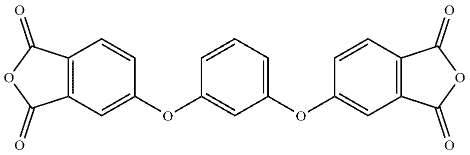 [SMILES: O=C1OC(=O)c2cc(Oc3cccc(Oc4ccc5c(c4)C(=O)OC5=O)c3)ccc21]

### Example 12: Synthesis of Dianhydride From N-methyl Nitrophthalimide and Bisphenol A

This example establishes the method of preparation of dianhydride from nitrophthalimide and bisphenol A without isolating the diimide.

A 250 ml three-neck round bottomed flask containing a magnetic stirrer bar is fitted with a thermocouple, a nitrogen inlet and a nitrogen outlet with a bubbler through a Dean-Stark trap. The flask is charged with bisphenol A (2.164 g, 0.005 moles, 1.00 equivalents) and DMSO (10 ml). To this stirring solution, sodium hydroxide (0.42 g, 0.0105 mol, 2.1 equivalent) added as 50% solution in water. The reaction flask is heated to 90° C. in oil bath until the salt formation is complete. 20 ml toluene is added to this mixture and the water toluene is azeotroped into the Dean-Stark trap. To the stirring dry reaction mixture at 100° C., N-methyl-4-nitrophthalimide (2.164 g, 0.0105 moles, 2.10 equivalents) is added and the stirring continued until the reaction is complete.

Once complete, the DMSO solution of the diimide reaction mixture is transferred into a 50 ml autoclave reactor with a magnetic stirrer bar. Acetic acid (10 g) and water (20 ml) are added to the solution. The reactor is sealed and heated at 190° C. and pressure of 200 psi while stirring. Once complete the reactor is cooled to room temperature. The reaction mixture is diluted with water (30 ml) to precipitate the resulting tetra acid. The aqueous slurry is centrifuged to obtain a solid which is dried under vacuum at room temperature.

The solid tetra acid is flushed with nitrogen and heated to 200° C. under vacuum to obtain the dianhydride of the formula (13),

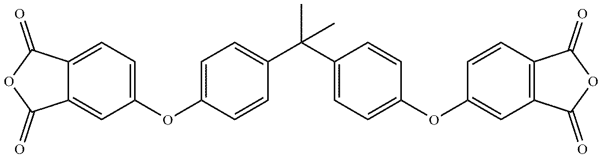 [SMILES: CC(C)(c1ccc(Oc2ccc3c(c2)C(=O)OC3=O)cc1)c1ccc(Oc2ccc3c(c2)C(=O)OC3=O)cc1]

### References

2019/0119240
2019/0092726
US 4,329,496
US 6,008,374
US 5,359,084
US 3,879,428
US 4,017,511
US 5,536,846
US 3,957,862
US 4,263,209
US 4,571,425
US 4,318,857
US 7,495,113
US 4,221,897
WO 2019/245898 A1
WO 2017/172593 A1
WO 2019/236536 A1
WO 2019/222077 A1
WO 2017/189293 A1
WO 2019/217257 A1

All publications, including patent documents and scientific articles, referred to in this application and the bibliography and attachments are incorporated by reference in their entirety for all purposes to the same extent as if each individual publication were individually incorporated by reference.

All headings and titles are for the convenience of the reader and should not be used to limit the meaning of the text that follows the heading, unless so specified.

What is claimed is:

1. A method of making a dianhydride, comprising:
a) combining bisphenol with halo or nitro N-substitued phthalimide and sodium hydride in a polar aprotic solvent to provide at least one mono-imide, at least one diimide, or a combination thereof in a solution;
b) isolating said diimide from said solution to form an isolate;
c) combining said isolated diimide with carboxylic acid and dimethylsulfoxide to provide at least one tetra acid, at least one triacid, at least one imide diacid, or a combination thereof;
d) precipitating said tetra acid, said triacid, said imide diacid, or a combination thereof to form a precipitate;
e) removing the sulfoxide, carboxylic acids, and other byproducts from said precipitate; and
f) converting said precipitate to the corresponding dianhydride;
wherein said halo and nitro N-substituted phthalimide is of formula 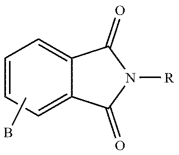
wherein said bisphenol is of formula 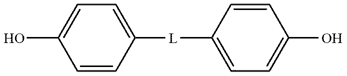
wherein said diimide is of the formula 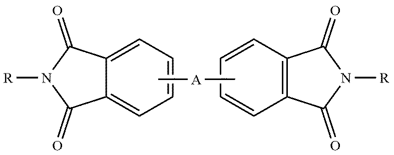
wherein said carboxylic acid is of formula X-COOH
wherein said sulfoxide is of formula J₂SO
wherein said tetra acid is of formula, 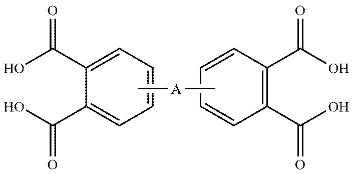
wherein said triacid is of formula, 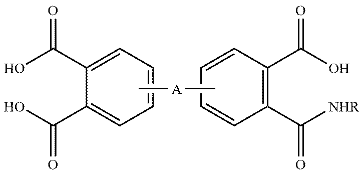
wherein said diacid imide is of the formula, 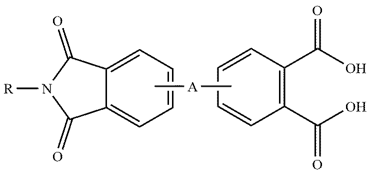
wherein said dianhydride is of the formula, 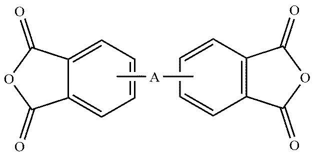
wherein in the forgoing formulas
B is —NO2, —Cl, —F, —Br, or —I;
A is —O—, —S—, —C(O)—, —SO₂—, —SO—, —C_{y}H_{2y}—
wherein y is an integer from 1 to 5 or
a halogenated derivative thereof or —O—E—O—,
wherein E is an aromatic C₆₋₂₄ monocyclic or polycyclic moiety optionally substituted with 1 to 6 of C₁₋₈ alkyl groups, 1 to 8 halogen atoms, or a combination comprising at least one of the foregoing;
wherein R is a monovalent C₁₋₁₃ organic group;
wherein X is aryl group, C₁₋₈ alkyl group, or preferably a methyl group.
wherein J is C₁-₈ alkyl group, or aryl group, preferably a methyl group.

2. The method of claim 1;
wherein said method comprises the reacting a halo- or nitro- N-substituted phthalimide with bisphenol and sodium hydride in polar aprotic solvent under condition to provide diimide which on reacting with a carboxylic acid and substituted or unsubstituted dimethyl sulfoxide in an aqueous medium under conditions to provide a reaction mixture.

3. The method of claim 1;
wherein in a) the reaction temperature is between about 0 to about 250° C.

4. The method of claim 1;
wherein in b) said isolating is by precipitating in acetic acid solution.

5. The method of claim 1;
wherein in c) the reaction temperature is between about 160 to about 250° C.

6. The method of claim 1;
wherein in c) the reaction pressure is between about 150 to about 300 psig, between about 170 to about 250 psig, or a combination thereof.

7. The method of claim 1;
wherein in d) said precipitating is performed in water.

8. The method of claim 1;
wherein in e) said removing is performed by filtering the precipitate.

9. The method of claim 1;
wherein E is 2,2-(4-phenylene)isopropylidene.

10. The method of claim 1;
wherein E is 4-phenylene-1,1′-biphenyl.

11. The method of claim 1;
wherein in c) the initial mass ratio of acetic acid to diimide is between about 1:1 to about 50:1, or about 1:1 to about 20:1, or about 1:1 to about 10:1.

12. The method of claims 1;
wherein in c) the initial mass ratio of dimethyl sulfoxide to diimide is between about 1:1 to about 50:1, or about 1:1 to about 20:1, or about 1:1 to about 10:1.

13. The method of claims 1;
wherein in c) the initial mass ratio of water to diimide is between about 1:1 to about 100:1, or about 2:1 to about 50:1, or about 2:1 to about 20:1.

14. The method of claim 1;
wherein in c) the reaction mixture further comprises the diimide, acetic acid with its derivatives, and dimethyl sulfoxide and its reaction and decomposition products.

15. The method of the claims 1;
wherein in d) the precipitation is done by adding into water.

16. The method of the claims 1;
wherein in d) the precipitation is done by cooling the reaction mixture to between about 5 to about 50° C.

17. The method of the claim 1;
wherein in d) the ratio of reaction mixture to water for precipitation is between about 1:0 to about 1:10.

18. The method of the claims 1;
wherein in f) the precipitate is heated at 180 to 250° C. under the reduced pressure of less than about 200 mm/Hg.

19. The method of any of the claims 1;
wherein in f) the reaction mixture is directly converted into dianhydride by heating at between about 180 to about 250° C. under the reduced pressure less than about 200 mm of Hg.

20. The method of claim 1;
wherein conversion of diimide to dianhydride is at least about 90%, preferably at least about 96%.

21. The method of any of claims 1;
wherein the diimide comprises 4,4′-bisphenol A-bis-N-methylphthalimide, 3.4′-bisphenol A-bis-N-methylphthalimide, 3,3′-bisphenol A-bis-N-methylphthalimide, or a combination comprising at least one of the foregoing; the diimide also comprises 4,4′-biphenol-N-methylphthalimide, 3,4′-biphenol-N-methylphthalimide, 3,3′-biphenol-N-methylphthalimide or a combination comprising at least one of the foregoing; the dianhydride comprises 4,4′-bisphenol A-bis-dianhydride, 3,4′-bisphenol A-bisdianhydride, 3,3′-bisphenol A-bis-dianhydride, or a combination comprising at least one of the forgoing; the dianhydride also comprises 4,4′-biphenol-bisanhydride, 3,4′-biphenol-bisanhydride, 3,3′-biphenol-bisanhydride or a combination comprising at least one of the forgoing.

22. The method of any claim 1;
wherein imide anhydride is present in an amount of less than about 10%, preferably less than about 4%, based on the total weight of the imide anhydride and dianhydride.

23. The method of any claims 1;
wherein the product dianhydride contains traces of diimide.

24. The method of any claims 1;
wherein the dianhydride contains the dimethyl sulfoxide and its derivatives as impurities.

25. The method of any claims 1;
wherein the dianhydride contains the acetic acid and its derivatives as impurities.

26. A method for manufacture of polyetherimide composition, the method comprising:
a) manufacturing a dianhydride in accordance with the method of claim 1;
b) polymerizing the dianhydride and a diamine to provide a polyetherimide composition.

27. A polyetherimide composition manufactured by the method of claim 26.
